(19) United States
(12) Patent Application Publication
(10) Pub. No.: US 2012/0014880 A1
(43) Pub. Date: Jan. 19, 2012

(54) METHOD FOR THE PREVENTION AND TREATMENT OF ESSENTIAL TREMOR BY REGULATING alpha1G T-TYPE CALCIUM CHANNEL OR BY T-TYPE CALCIUM CHANNEL BLOCKERS

(75) Inventors: Daesoo Kim, Daejeon (KR); Hyeyeon Park, Seoul (KR); Hee-Sup Shin, Kyoungki-do (KR); Young-Gyun Park, Daejeon (KR)

(73) Assignee: Korea Advanced Institute of Science and Technology

(21) Appl. No.: 13/243,325

(22) Filed: Sep. 23, 2011

Related U.S. Application Data
(62) Division of application No. 12/120,163, filed on May 13, 2008.

(30) Foreign Application Priority Data
Nov. 15, 2007 (KR) 10-2007-0116655

(51) Int. Cl. A61K 49/00; C12Q 1/02; A61P 25/14

(52) U.S. Cl. 424/9.2; 435/29

(57) ABSTRACT

The present invention relates to a method for the prevention and treatment of essential tremor by blocking α1G T-type calcium channel, a preventive and therapeutic agent for essential tremor containing the α1G T-type calcium channel blocker as an active ingredient, and a screening method of a preventive and therapeutic agent for essential tremor by investigating α1G T-type calcium channel blocking activity. More precisely, the present invention relates to a method for the prevention and treatment of essential tremor by using α1G T-type calcium channel blocker, for which the inventors confirmed that the α1G T-type calcium channel knock out mice (α1G−/−) had resistance against essential tremor and when the T-type channel blocker was administered to the wild type mice (α1G+/+), they gained resistance against essential tremor.

### CROSS REFERENCE TO RELATED APPLICATIONS

This is a divisional of U.S. patent application Ser. No. 12/120,163, filed May 13, 2008, which claims the benefit under 35 U.S.C. §119 of Korean Patent application number KR 10-2007-0116655, filed on Nov. 15, 2007, both of which are herein incorporated by reference in their entirety.

### TECHNICAL FIELD

The present invention relates to a method for the prevention and treatment of essential tremor by blocking α1G T-type calcium channel, a preventive and therapeutic agent for essential tremor containing the α1G T-type calcium channel blocker as an active ingredient, and a screening method of a preventive and therapeutic agent for essential tremor by investigating α1G T-type calcium channel blocking activity.

### BACKGROUND ART

Essential tremor is a kind of motor disorder characterized by the symptom of regular tremor of a part of body. Essential tremor is a hereditary disease, which is inherited through autosomal dominant inheritance, suggesting that essential tremor patient has family history thereof. Thus, essential tremor is also called familial tremor. Essential tremor is generally expressed as a neurological single symptom, and is comparatively common motor disorder that is found in 30˜40 out of 1,000 people. Tremor caused by the disease is different from that caused by Parkinson's disease shown in rest or systemic or local myotinia, although it can be accompanied with these. It is also distinguished from cerebellar tremor. The diagnostic characteristic of essential tremor is consistent or transient tremor in hands, head, and voice, which is shown by the changes of physical condition and kinesis. However, these symptoms are not detected in stable phase unless it is progressed severely. No other neurological disorders in relation to systemic or neuronal disease are caused. It is easy to diagnose this disorder by its familial tendency and sometimes drinking alcohol can reduce tremor temporarily. The cause of essential tremor has not been disclosed and no precise animal models have been established, yet. Even though it depends on each individual, essential tremor starts with minute tremor in one side or in both sides of body and progresses slowly.

Voltage dependent calcium channel plays a role in increasing intracellular calcium concentration by active neurons (Tsien, R. W., Annu Rev Physiol 45, 341-358, 1983). This channel is classified by the level of voltage dependence into high-voltage dependent and low-voltage dependent channels (Tsien, R. W. et al., Trends Neurosci 18, 52-54, 1995). T-type calcium channel is the representative low-voltage dependent calcium channel, which exists as Cav3.1 (α1G), 3.2 (α1H) and 3.3 (α1I) in mammals according to al subunit (Perez-Reyes, E., Physiol Rev 83, 117-161, 2003). α1G calcium channel is involved in the generation of neuronal burst firings in thalamic nuclei and its important pathological functions have been recently disclosed (Kim, D. et al., Science 302, 117-119, 2003; Kim, D. et al., Neuron 31, 35-45, 2001).

The conventional method for treating essential tremor is taking alcohol by drinking or alcohol compound such as octanol and propanol; taking inhibitory drugs such as GABA receptor agonist; and brain surgery including thalamectomy or deep-brain stimulation. However, the GABA agonist or alcohol compound inhibits the various functions of nerve system in addition to tremor and moreover causes serious side-effect including sleep induction, etc. And the dangerous brain surgery such as thalamectomy or deep-brain stimulation has to be last resort.

The present inventors have studied on essential tremor using α1G T-type calcium channel knock-out mice (α1G−/−) and confirmed that the α1G T-type calcium channel knock-out mice (α1G−/−) had resistance against essential tremor and a wild-type mouse (α1G+/+) also demonstrated resistance against essential tremor when they were administered with T-type channel blockers. Based on that, the present inventors completed this invention by confirming that essential tremor can be prevented and treated by blocking α1G T-type calcium channel.

### DISCLOSURE

### Technical Problem

It is an object of the present invention to provide a method for the prevention and treatment of essential tremor by blocking α1G T-type calcium channel, a preventive and therapeutic agent for essential tremor containing the α1G T-type calcium channel blocker as an active ingredient, and a screening method of a preventive and therapeutic agent for essential tremor by investigating α1G T-type calcium channel blocking activity.

### Technical Solution

To achieve the above object, the present invention provides a method for the prevention and treatment of essential tremor by blocking α1G T-type calcium channel.

The present invention also provides a preventive and therapeutic agent for essential tremor containing the α1G T-type calcium channel blocker as an active ingredient.

The present invention further provides a method for treating essential tremor containing the step of administering the α1G T-type calcium channel blocker to a subject.

The present invention also provides a method for preventing essential tremor containing the step of administering the α1G T-type calcium channel blocker to a subject.

The present invention also provides a use of the α1G T-type calcium channel blocker for the production of a preventive and therapeutic agent for essential tremor.

The present invention also provides a screening method of the preventive and therapeutic agent for essential tremor, comprising the following steps:

1) The cells expressing α1G T-type calcium channel stably are divided into two groups and the experimental group is treated with samples and the control group is not treated with samples, followed by culture;

2) The activity of α1G T-type calcium channel expressed in the cells of step 1) is measured by whole cell patch clamp; and

3) The sample blocking the activity of α1G T-type calcium channel in the experimental group, compared with the control, is selected.

In addition, the present invention provides a screening method of the preventive and therapeutic agent for essential tremor, comprising the following steps:

1) The cells expressing α1G T-type calcium channel stably are treated with samples and then the activity of α1G T-type calcium channel is measured to screen a α1G T-type calcium channel blocker;

2) The subject induced with essential tremor by the administration of an essential tremor inducing material is administered with the α1G T-type calcium channel blocker screened in step 1);

3) Symptoms of essential tremor are investigated in the subject of step 2); and

4) The substance relieving the symptoms of essential tremor in the experimental group, compared with the control group of step 3) is selected.

### Advantageous Effect

The present invention demonstrated that the α1G T-type calcium channel knock-out mouse (α1G−/−) had resistance against essential tremor and when the T-type channel blocker was administered to a wild-type mouse (α1G+/+), this mouse also exhibited resistance against essential tremor. So, this invention disclosed that α1G T-type channel is an important target for treating essential tremor. Therefore, based on the disclosure of the exact mechanism of α1G T-type calcium channel, it can be achieved the development of a therapeutic agent for essential tremor without side effects generally observed when the conventional T-type channel blockers are administered and a method thereof.

### DESCRIPTION OF DRAWINGS

The application of the preferred embodiments of the present invention is best understood with reference to the accompanying drawings, wherein:

FIG. 1 is a diagram illustrating the result of tremor detection by using movement artifact of electroenphalograph (EEG) with α1G−/−transgenic mice after the intraperitoneal injection of tremor inducing agents.

FIG. 2 is a diagram illustrating the resistance of α1G−/−mice against essential tremor, measured by using tremor quantification apparatus after the intraperitoneal injection of tremor inducing agents:

(A) wild type;

(B) α1G−/−;

(C) strength detected under the frequency of 10-18 Hz, characteristic frequency of essential tremor; and

(D) time point of development of essential tremor, duration, strength and maximum frequency.

FIG. 3 is a diagram illustrating the effect of mibefradil on harmaline induced tremor according to the administration pathways:

(A) Mibefradil that could not pass through blood-brain barrier was injected intraperitoneally; and

(B) Mibefradil was directly inserted into inferior olive (TO) of the brain.

FIGS. 4A and B are diagrams illustrating the effect of ethosuximide on harmaline induced tremor.

### BEST MODE

Hereinafter, the present invention is described in detail.

The present invention provides a method for the prevention and treatment of essential tremor by blocking α1G T-type calcium channel.

The present invention also provides a preventive and therapeutic agent for essential tremor containing the α1G T-type calcium channel blocker as an active ingredient.

The α1G T-type calcium channel blocker herein is preferably selected from the group consisting of mibefradil and its derivatives, suximide derivatives including ethosuximide, efonidipine, trivalent metal ions, U-92032 (7-[[4-[bis(4-fluorophenyl)methyl]-1-piperazinyl]methyl]-2-[(2-hydroxyethyl)amino]-4-(1-methylethyl)-2,4,6-cycloheptatrien-1-one), penfluridol, fluspirilene and valporate, but not always limited thereto and any substance blocking T-type calcium channel can be used (Masumiya et al., Life Sci., 2000, 68(3): 345-51; Mlinar et al., J. Physiol., 1993, 469: 639-52; Xu and Lee, J. Pharmacol. Exp. Ther., 1994, 268: 1135-1142; Enyeart et al., Mo; Macdonald and Kelly, Epilepsia, 1994, 35 Suppl 4: S41-50).

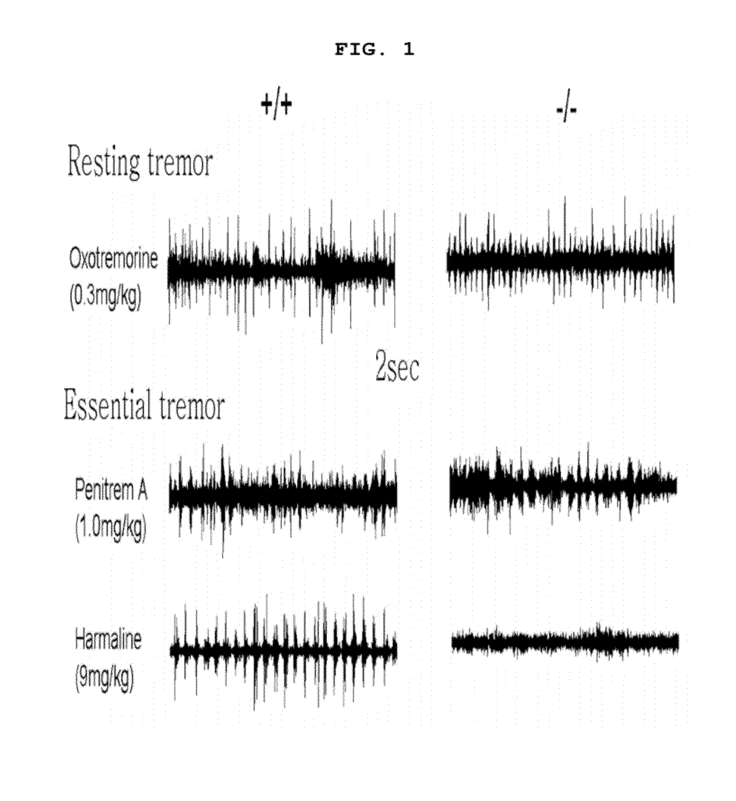

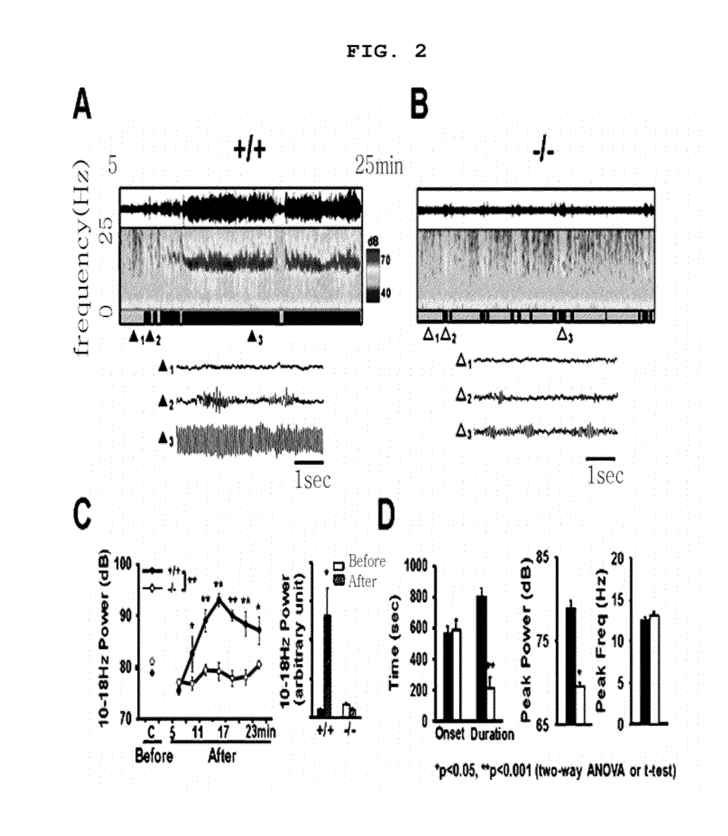

To investigate whether or not α1G T-type calcium channel was related to essential tremor, the present inventors prepared α1G T-type calcium channel knock-out (α1G−/−) mice in which essential tremor was induced and observed resistance against essential tremor. Particularly, to prepare the α1G T-type calcium channel knock-out (α1G−/−) mice, a fertilized egg having α1G+/−genotype (International Depositary Authority, Korean Collection for Type Cultures, Korea Research Institute of Bioscience and Biotechnology, Accession No: KCTC 10086 BP) was transplanted into a surrogate mother mouse, resulting in heterozygote transgenic mice having α1G+/−genotype. Then, the heterozygote transgenic female mouse and male mouse were mated. Both wile-type and the α1G−/−mice were intraperitoneally administered with tremor inducing agents, oxotremorine, penitrem A and harmaline, to induce tremor, followed by measuring tremor development by using electroenphalograph (EEG) and tremor quantification apparatus. From the investigation on the development of tremor using the electroenphalograph (EEG), it was confirmed that the α1G T-type calcium channel knock-out mouse was induced with oxotremorine inducing static tremor, the symptom of Parkinson's disease tremor, like a wild-type mouse. This mouse was also induced with penitrem A inducing essential tremor, where penitrem A is one of the essential tremor inducers but develops tremor regardless of the presence of or inferior olivary nucleus. However, the α1G T-type calcium channel knock-out mouse showed resistance against harmaline inducing essential tremor, where inferior olivary nucleus is required to develop. From the investigation on the development of essential tremor using the tremor quantification apparatus, it was confirmed that the α1G T-type calcium channel knock-out mouse (α1G−/−) demonstrated high resistance against harmaline induced tremor, compared with a wild-type mouse (α1G+/+). The above results indicate that the α1G T-type calcium channel knock-out mouse (α1G−/−) has selective resistance against inferior olivary nucleus dependent essential tremor and thus α1G T-type calcium channel can be an effective target for the treatment of essential tremor (see FIGS. 1 and 2).

To investigate whether or not the inhibition of α1G T-type calcium channel could be a method for inhibiting essential tremor, essential tremor was induced in a wild-type mouse (α1G+/+) by using harmaline, and then mibefradil and ethosuximide, known as the conventional T-type channel blockers, were treated thereto, followed by measuring the inhibition of essential tremor by using tremor quantification apparatus. As a result, injection of mibefradil in the brain resulted in the inhibition of essential tremor, but intraperitoneal injection of the same resulted in no inhibition effect. In the meantime, intraperitoneal injection of ethosuximide resulted in the inhibition of essential tremor. The above results seemed to be because that mibefradil could not pass through blood-brain barrier. Thus, mibefradil derivatives modified with their basic structures to pass through blood-brain barrier or ethosuximide and other α1G T-type channel blockers can be used as a therapeutic agent for essential tremor.

The conventional therapeutic agents including alcohol enhance GABA receptor and thereby get neuronal activity in the whole brain to be dull, whereas the α1G T-type channel of the present invention is activated by GABA receptor. It is also very easy to control selectively the down stream of GABA receptor, so the α1G T-type channel can be easily regulated with less side effects.

In this invention, motor behavior of the α1G T-type calcium channel knock-out (α1G−/−) mice were observed. As a result, there was no abnormal walking or motor learning. Therefore, it was confirmed that the inhibition of α1G T-type calcium channel can be a method for treating essential tremor without side effects.

The present invention also provides a method for treating essential tremor containing the step of administering the α1G T-type calcium channel blocker to a subject.

The present invention also provides a method for preventing essential tremor containing the step of administering the α1G T-type calcium channel blocker to a subject.

The present invention also provides a use of the α1G T-type calcium channel blocker for the production of a preventive and therapeutic agent for essential tremor.

The α1G T-type calcium channel blocker is preferably selected from the group consisting of mibefradil and its derivatives, suximide derivatives including ethosuximide, efonidipine, trivalent metal ions, U-92032 (7-[[4-[bis(4-fluorophenyl)methyl]-1-piperazinyl]methyl]-2-[(2-hydroxyethyl)amino]-4-(1-methylethyl)-2,4,6-cycloheptatrien-1-one), penfluridol, fluspirilene and valporate, but not always limited thereto and any T-type calcium channel blocker can be used (Masumiya et al., Life Sci., 2000, 68(3): 345-51; Mlinar et al., J. Physiol., 1993, 469: 639-52; Xu and Lee, J. Pharmacol. Exp. Ther., 1994, 268: 1135-1142; Enyeart et al., Mo; Macdonald and Kelly, Epilepsia, 1994, 35 Suppl 4: S41-50).

The present invention also provides a screening method of the preventive and therapeutic agent for essential tremor, comprising the following steps:

1) The cells expressing α1G T-type calcium channel stably are divided into two groups and the experimental group is treated with samples and the control group is not treated with samples, followed by culture;

2) The activity of α1G T-type calcium channel expressed in the cells of step 1) is measured by whole cell patch clamp; and

3) The sample blocking the activity of α1G T-type calcium channel in the experimental group, compared with the control, is selected.

In the screening method of the present invention, the cells expressing α1G T-type calcium channel stably of step 1) are exemplified by HEK293 cells described in Korean Patent No. 10-519693 or transformed HEK293 cells transformed with the plasmid containing human Kir2.1 gene, but not always limited thereto. The transformed HEK293 cells were deposited under the Accession No. of KCTC10519BP.

In the screening method of the present invention, the sample of step 1) can be selected from the group consisting of nucleic acids, proteins, other extracts or natural substances that are expected to have a potential as a T-type calcium channel blocker or randomly selected.

In the screening method of the present invention, the method for measuring the activity of α1G T-type calcium channel of step 2) is described in Korean Patent No. 10-519693.

In addition, the present invention provides a screening method of the preventive and therapeutic agent for essential tremor, comprising the following steps:

1) The cells expressing α1G T-type calcium channel stably are treated with samples and then the activity of α1G T-type calcium channel is measured to screen the α1G T-type calcium channel blocker;

2) The subject induced with essential tremor by the administration of an essential tremor inducing material is administered with the α1G T-type calcium channel blocker screened in step 1);

3) Symptoms of essential tremor are investigated in the subject of step 2); and

4) The substance relieving the symptoms of essential tremor in the experimental group, compared with the control group of step 3) is selected.

In the screening method of the present invention, the cells expressing α1G T-type calcium channel stably of step 1) are exemplified by HEK293 cells described in Korean Patent No. 10-519693 or transformed HEK293 cells transformed with the plasmid containing human Kir2.1 gene, but not always limited thereto. The transformed HEK293 cells were deposited under the Accession No. of KCTC10519BP.

In the screening method of the present invention, the essential tremor inducing agent of step 2) is preferably hamaline depending on inferior olivary nucleus of the brain, but not always limited thereto.

In the screening method of the present invention, symptoms of essential tremor of step 3) are involuntary, regular movements of hands, arms, head, face, vocal band, trunk or legs. The symptoms can be checked by movement artifact of electroenphalograph (EEG) or tremor quantification apparatus, but not always limited thereto. The tremor quantification apparatus herein is to detect tremor of the mouse, in which stainless test cage is hung on the support and the bottom of the cage is attached with accelerometer connected with electric wire to analog-digital converter so that the generated analog signals can be converted into digital signals recognizable by computer. Thereby, tremor of the mouse can be measured by analyzing the digital signals recognized by computer. In this invention, for 20 minutes from 5 minutes after the drug injection, oscillation was observed and the strength of oscillation was calculated over the time and frequency through Fourier Transformation. When the strength of oscillation for 5 seconds under the frequency of 10-18 Hz, that is the characteristic frequency of harmaline induced tremor, was at least 65 dB, it was diagnosed as tremor and the time point of break (onset), duration, strength, and the maximum frequency were all measured and compared between the wild type mice and the transgenic mice. The method for measuring tremor is developed on the basis of described method in the following references (Long M A. et al., J. Neurosci. 2002 Dec. 15, 22(24):10898-905; Milner, T E. et al., J. Neurophysiol. 1995 June, 73(6):2568-77).

The test samples capable of blocking α1G T-type calcium channel or suppressing essential tremor, detected by the screening method of the present invention, can be a candidate for the preventive and therapeutic agent for essential tremor.

The candidate for the preventive and therapeutic agent for essential tremor can be a leading compound for the development of a therapeutic agent for essential tremor and this leading compound can be modified in its structure or optimized in order to bring the effect of blocking α1G T-type calcium channel or suppressing essential tremor, leading to the development of a novel preventive and therapeutic agent for essential tremor.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Generation and Care of α1G−/−Transgenic Mice

### <1-1> Generation of α1G−/−Transgenic Mice

The present inventors generated transgenic mice having α1G−/−genotype by using the fertilized egg (International Depositary Authority, Korean Collection for Type Cultures, Korea Research Institute of Bioscience and Biotechnology, Accession No: KCTC 10086 BP) having α1G+/−genotype of T-type calcium channel. Particularly, the fertilized egg having α1G+/−genotype was transplanted into a surrogate mother to produce heterozygote transgenic mice having α1G+/−genotype. Then, the heterozygote transgenic female mouse and male mouse were mated to generate homozygote transgenic mice having α1G−/− genotype.

### <1-2> Animal Care

All the mice were raised in an animal facility where temperature and humidity were regulated, water and food were provided freely, light condition was set at 12 h day/12 h night and the day begins at 8 am. F2 female and male mice at 8-15 weeks were used for experiment.

### Example 2

### Investigation of Resistance of α1G−/−Transgenic Mice Against Tremor Inducing Agents

The α1G−/−transgenic mice generated in Example 1 and the wild-type mice were administered with the tremor inducing agents, oxotremorine (0.3 mg/kg), penitrem A (1.0 mg/kg) and harmaline (9 mg/kg) respectively by intraperitoneal injection to induce tremor. Tremor was investigated by using movement artifact of electroenphalograph (EEG) and tremor quantification apparatus designed by the present inventors. The tremor quantification apparatus had a stainless test cage (15×15×20 cm) hanging on the support and an accelerometer on the bottom of the cage. The accelerometer was connected to analog-digital converter (Digidata1320, Axon instrument Inc) by electric wire so that the generated analog signals could be converted into digital signals recognizable by computer. Thereby, tremor of the mouse was measured by analyzing the digital signals recognized by computer. The accelerometer was quipped to the test cage where the animals were located, and the test cage was hung in the air. The level of tremor of the animal was quantified by measuring the oscillation of the test cage. In this experiment, for 20 minutes from 5 minutes after the drug injection, oscillation was observed and the strength of oscillation was calculated over the time and frequency through Fourier Transformation. When the strength of oscillation for 5 seconds under the frequency of 10-18 Hz, that is the characteristic frequency of harmaline induced tremor, was at least 65 dB, it was diagnosed as tremor and the time point of development of tremor (onset), duration, strength, and the maximum frequency were all measured and compared between the wild type mice and the knockout mice.

From the investigation on the development of essential tremor using movement artifact of the electroenphalograph (EEG), it was confirmed that the α1G−/−mouse was induced with oxotremorine inducing static tremor, the symptom of Parkinson's disease tremor, like a wild-type mouse. This mouse was also induced with penitrem A inducing essential tremor, where penitrem A is one of the essential tremor inducers but develops tremor regardless of the presence of or inferior olivary nucleus. However, the α1G−/−mouse showed resistance against harmaline inducing essential tremor, where inferior olivary nucleus is required to develop (FIG. 1).

From the result of investigation of resistance against essential tremor of the α1G−/−mouse by using the tremor quantification apparatus, it was confirmed that the α1G−/−mouse demonstrated resistance against harmaline inducing essential tremor, compared with the wild-type mouse (FIG. 2).

### Example 3

### Inhibition Effect on Essential Tremor by T-Type Channel Blockers

### <3-1> Effect of Mibefradil

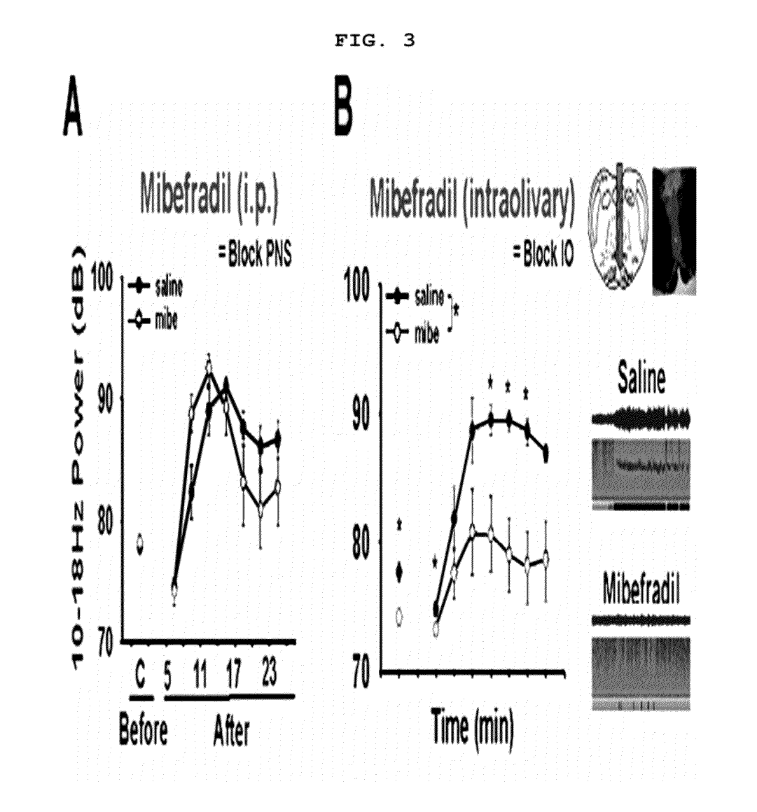

The wild-type mouse (α1G+/+) was administered with 10 mg/kg of mibefradil by intraperitoneal injection 30 minutes before harmaline injection to investigate the therapeutic effect of mibefradil on harmaline induced tremor. In the meantime, osmotic pump (Model 1002, 0.25 ul/hour, Alzet) containing 20 mM of mibefradil was inserted into the brain of the wild-type mouse (α1G+/+). Two days later, the therapeutic effect on harmaline induced tremor was investigated by using the tremor quantification apparatus of Example 2. As a result, when mibefradil incapable of passing through blood-brain barrier was administered by intraperitoneal injection, tremor inhibiting effect was not detected. In the meantime, when mibefradil was directly injected into inferior olive (IO) of the brain, tremor inhibiting effect was observed (FIG. 3).

### <3-2> Effect of Ethosuximide

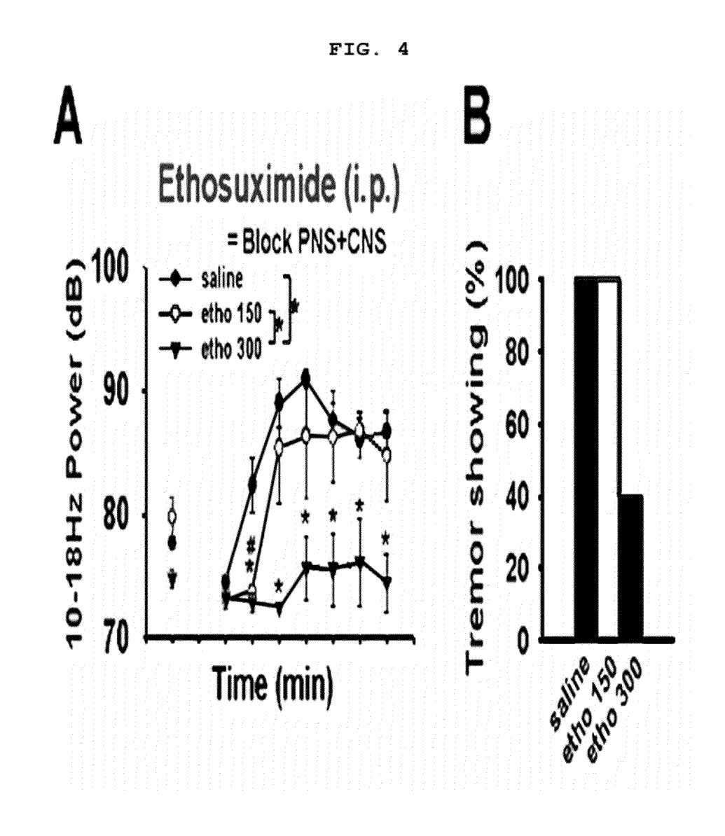

The wild-type mice (α1G+/+) were administered with 150 mg/kg and 300 mg/kg of ethosuximide by intraperitoneal injection 30 minutes before harmaline injection to investigate the therapeutic effect of ethosuximide on harmaline induced tremor. For the investigation, the tremor quantification apparatus of Example 2 was used. As a result, when ethosuximide was administered by intraperitoneal injection (respectively by 150 mg/kg and 300 mg/kg), tremor inhibiting effect was observed at both concentrations of 150 mg/kg and 300 mg/kg. In particular, at 300 mg/kg of the ethosuximide concentration, only 40% of the mice showed tremor and 60% of the mice did not exhibit oscillation, symptom of tremor (FIG. 4).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of screening for a therapeutic agent for essential tremor comprising the following steps:
1) dividing cells stably expressing α1G T-type calcium channel into an experimental group and a control group and the experimental group is treated with a sample and the control group is not treated the sample, followed by culture;
2) measuring activity of α1G T-type calcium channel expressed in the cells of step 1) by whole cell patch clamp; and
3) selecting the sample blocking the activity of α1G T-type calcium channel in the experimental group, compared with the control, thereby identifying a therapeutic agent for essential tremor.

2. The method according to claim 1, wherein the cells stably expressing α1G T-type calcium channel of step 1) are HEK293 cells or HEK293 cells transformed with a plasmid containing human Kir2.1 gene.

3. The method according to claim 2, wherein the transformed HEK293 cells are those cells deposited under the Accession No. KCTC10519BP.

4. A method of screening for a therapeutic agent for essential tremor comprising the following steps:
1) treating cells stably expressing α1G T-type calcium channel with a sample and measuring activity of α1G T-type calcium channel, thereby identifying an α1G T-type calcium channel blocker;
2) inducing essential tremor in a subject by administration of an essential tremor inducing agent and administering to the subject with the α1G T-type calcium channel blocker identified in step 1);
3) determining symptoms of essential tremor in the subject of step 2); and
4) selecting the substance relieving the symptoms of essential tremor in the subject compared with a control group, thereby identifying a therapeutic agent for essential tremor.

5. The method according to claim 4, wherein the cells stably expressing α1G T-type calcium channel of step 1) are HEK293 cells or HEK293 cells transformed with a plasmid containing human Kir2.1 gene.

6. The method according to claim 5, wherein the transformed HEK293 cells are those cells deposited under the Accession No. KCTC10519BP.

7. The method according to claim 4, wherein the essential tremor inducing agent of step 2) is harmaline depending on inferior olivary nucleus of the brain.

8. The method according to claim 4, wherein the symptoms of essential tremor of step 3) are involuntary, regular movements of hands, arms, head, face, vocal band, trunk or legs.

9. The method according to claim 4, wherein determining symptoms of essential tremor of step 3) is performed by using electroenphalograph (EEG) or tremor quantification apparatus.
